(19) United States
(12) Patent Application Publication
(10) Pub. No.: US 2005/0191657 A1
(43) Pub. Date: Sep. 1, 2005

(54) Stringency modifiers in hybridization assays

(76) Inventors: David M. Demorest, Soquel, CA (US); Carolyn Gonzalez, San Mateo, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/993,220

(22) Filed: Nov. 18, 2004

Related U.S. Application Data
(60) Provisional application No. 60/523,368, filed on Nov. 18, 2003.

(51) Int. Cl. C12Q 1/68; C07H 21/04

(52) U.S. Cl. 435/6; 536/24.3; 536/25.32

(57) ABSTRACT

Methods of modifying stringency in hybridization assays are provided. Kits for modifying stringency in hybridization assays are also provided.

This application claims the benefit of U.S. Provisional Application No. 60/523,368, filed Nov. 18, 2003, which is incorporated by reference herein in its entirety for any purpose.

### I. FIELD

The invention relates to methods and compositions in hybridization assays.

### II. BACKGROUND

Various molecular biology methods utilize hybridization of complementary DNA and/or RNA sequences. Such methods include, for example, Northern and Southern blots, dot blots, slot blots, array assays (including microarrays), ligation assays, oligonucleotide ligation assays combined with polymerase chain reactions (OLA-PCR), and various solution phase methods, including, but not limited to, those employed by a variety of diagnostic tests. After hybridization (in which DNA/DNA, RNA/DNA, and/or RNA/RNA complexes may be formed through nucleotide base-pairing) the presence or absence of hybridization may be detected by any of a variety of methods, including, for example, chemical fluorometric, radioactive or enzymatic labeling of a probe or of a target nucleic acid, or by mass spectrometry.

Nucleic acids may hybridize if they comprise complementary sequences. Under certain conditions, nucleic acids may hybridize even if there are mismatched bases. The ability of nucleic acids with certain mismatched bases to hybridize may be manipulated by changing the conditions of the hybridization. Certain higher stringency conditions result in less tolerance for mismatched bases. Thus, higher stringency conditions result in a higher ratio of the amount of hybridization of sequences with fewer mismatches to the amount of hybridization of sequences with more mismatches than lower stringency conditions. Conversely, lower stringency conditions allow hybridization of sequences with more mismatches, thereby resulting in a lower ratio of the amount of hybridization of sequences with fewer mismatches to the amount of hybridization of sequences with more mismatches. Therefore, higher stringency conditions result in higher specificity hybridization, and lower stringency conditions result in lower specificity hybridization.

For a particular hybridization assay, stringency conditions are typically optimized for the results desired. In certain instances, if the stringency is too low, hybridization among sequences containing an undesirable number of mismatched base pairs may result. In certain instances, if the stringency is too high, sequences with a desired number of mismatched base pairs may not hybridize sufficiently.

An example of a variable that may affect stringency in a hybridization assay is temperature. Typically, increasing the temperature of a hybridization assay increases the stringency. Another method for altering stringency is to add one or more stringency modifiers to the hybridization assay. For example, formamide has been used for this purpose. Similar to the effect of temperature, the presence of certain chemical stringency modifiers may result in higher stringency.

### III. SUMMARY

In certain embodiments, methods of modifying stringency in a hybridization assay are provided comprising using at least one stringency modifier in a hybridization assay, wherein the at least one stringency modifier is selected from: 2-pyrrolidinone; an N-alkyl-pyrrolidinone; delta-valerolactam; and epsilon-caprolactam.

In certain embodiments, kits for performing an array assay are provided comprising a hybridization buffer comprising at least one stringency modifier selected from: 2-pyrrolidinone; an N-alkyl-pyrrolidinone; delta-valerolactam; and epsilon-caprolactam; and an array.

### IV. DETAILED DESCRIPTION OF CERTAIN EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. PCT International Publication Nos. WO 01/092579 A3, published Dec. 6, 2001, WO 03/054511 A2, published Jul. 3, 2003, and WO 2004/040020 A1, published May 13, 2004; and U.S. Patent Application Publication Nos. US 2002/0137047 A1, published Sep. 26, 2002, US 2002/0110828 A1, published Aug. 15, 2002, US 2003/0082572 A1, published May 1, 2003, US 2003/0165935 A1, published Sep. 4, 2003, US 2004/0101843 A1, published May 27, 2004, US 2004/0214196 A1, published Oct. 28, 2004, and US 2004/0121371 A1, published Jun. 24, 2004, are hereby expressly incorporated by reference in their entirety for any purpose.

A. Certain Definitions

The term “nucleotide base”, as used herein, means a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-d ihydrothymine, 5,6-d ihyd rou racil, pyrazolo[3,4-D]pyrimid ines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, means a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C 1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352;, and WO 99/14226). Exemplary LNA sugar analogs within a polynucleotide include, but are not limited to, the structures: 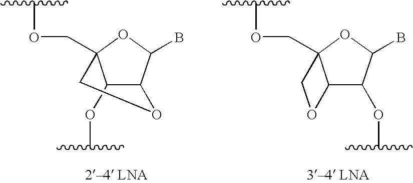
where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula: 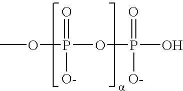
where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” means a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

The term “nucleotide analog”, as used herein, means embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphoroth ioates, phosphorod ithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

As used herein, the terms “polynucleotide”, “oligonucleotide”, and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. nucleic acids typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 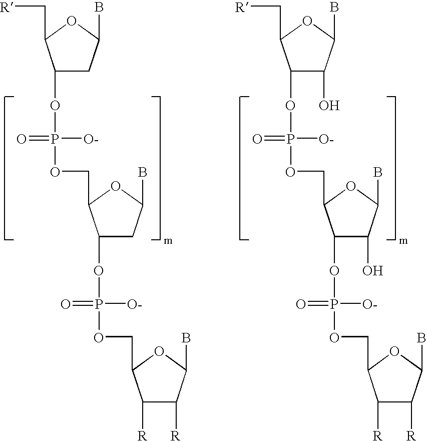
wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl or (C5-C14) aryl, or two adjacent Rs are taken together to form a bond such that the ribose sugar is 2′,3′-didehydroribose; and each R′ is independently hydroxyl or 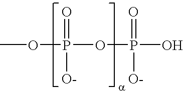
where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

The term “complement” means the compatibility of two sequences and their ability to match and form a hybrid. Thus, a target nucleic acid can be said to be a complement of the probe.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, polynucleotides, oligonucleotides, probes, primers, primer-specific portions, target-specific portions, addressable portions, and oligonucleotide link elements.

In this application, a statement that one sequence hybridizes or binds to another sequence encompasses situations where the entirety of both of the sequences hybridize or bind to one another, and situations where only a portion of one or both of the sequences hybridizes or binds to the entire other sequence or to a portion of the other sequence. Here, the term “sequence” encompasses, but is not limited to, nucleic acid sequences, polynucleotides, oligonucleotides, probes, primers, primer-specific portions, target-specific portions, addressable portions, and oligonucleotide link elements.

In this application, a statement that one sequence is complementary to another sequence encompasses situations in which the two sequences have mismatches. Here, the term “sequence” encompasses, but is not limited to, nucleic acid sequences, polynucleotides, oligonucleotides, probes, primers, primer-specific portions, target-specific portions, addressable portions, and oligonucleotide link elements. Despite the mismatches, the two sequences should selectively hybridize to one another under appropriate conditions.

The term “selectively hybridize” means that, for particular identical sequences, a substantial portion of the particular identical sequences hybridize to a given desired sequence or sequences, and a substantial portion of the particular identical sequences do not hybridize to other undesired sequences. A “substantial portion of the particular identical sequences” in each instance refers to a portion of the total number of the particular identical sequences, and it does not refer to a portion of an individual particular identical sequence. In certain embodiments, “a substantial portion of the particular identical sequences” means at least 90% of the particular identical sequences. In certain embodiments, “a substantial portion of the particular identical sequences” means at least 95% of the particular identical sequences. In certain embodiments, conditions are established so as to distinguish between a desired and an undesired sequence, using a detection system for the particular embodiment.

In certain embodiments, the number of mismatches that may be present may vary in view of the complexity of the composition. Thus, in certain embodiments, fewer mismatches may be tolerated in a composition comprising DNA from an entire genome than a composition in which fewer DNA sequences are present. For example, in certain embodiments, with a given number of mismatches, a probe may more likely hybridize to undesired sequences in a composition with the entire genomic DNA than in a composition with fewer DNA sequences, when the same hybridization conditions are employed for both compositions. Thus, that given number of mismatches may be appropriate for the composition with fewer DNA sequences, but fewer mismatches may be more optimal for the composition with the entire genomic DNA.

In certain embodiments, sequences are complementary if they have no more than 20% mismatched nucleotides. In certain embodiments, sequences are complementary if they have no more than 15% mismatched nucleotides. In certain embodiments, sequences are complementary if they have no more than 10% mismatched nucleotides. In certain embodiments, sequences are complementary if they have no more than 5% mismatched nucleotides.

The term “stringency” means conditions that affect the tolerance for mismatches in a hybridization assay. Less stringent conditions allow more hybridization of nucleic acids comprising mismatched bases. More stringent conditions allow less hybridization of nucleic acids comprising mismatched bases. Thus, under more stringent conditions, there is a greater amount of hybridization of nucleic acids with fewer mismatched bases relative to hybridization of nucleic acids with more mismatched bases, when compared to less stringent conditions.

The term “stringency modifier” means a compound that affects the stringency of a hybridization assay.

The terms “2-pyrrolidinone” and “2-pyrrolidone” are synonyms and mean compounds with the structure: 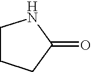
(CA Registry # 616-45-5) (empirical formula: C₄H₇NO), which may be obtained from chemical suppliers such as Aldrich (P/N P7,437-0) or Fluka (P/N 83300).

The term “N-alkyl pyrrolidinones” means compounds with the structure: 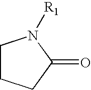
where R₁ is any alkyl group.

The term “delta-valerolactam” and “2-piperidone” are synonyms and mean compounds with the structure:

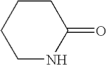

The term “epsilon-caprolactam”; “2-oxohexamethylenimine”; “1,6-hexanolactam”; and “6-hexanelactam,” are synonyms and mean compounds with the structure:

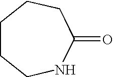

The term “stringency modifiers of the present invention” comprises; 2-pyrrolidinone, N-alkyl pyrrolidinones, Delta-valerolactam, and Epsilon-caprolactam and compounds that are structurally related to 2-pyrrolidinone, N-alkyl pyrrolidinones, Delta-valerolactam, and Epsilon-caprolactam.

A “target” or “target nucleic acid”, as used herein, comprises a specific nucleic acid sequence that can be distinguished by a probe. In certain embodiments, a target nucleic acid is naturally occurring. In certain embodiments, a target nucleic acid comprises synthetic molecules.

“Probes”, as used herein, comprise oligonucleotides that comprise a specific portion that is designed to hybridize in a sequence-specific manner with a complementary region on a specific nucleic acid, e.g., a target nucleic acid. In certain embodiments, the specific portion of the probe may be specific for a particular sequence, or alternatively, may be degenerate, e.g., specific for a set of sequences.

The term “label” means any molecule or group of molecules that can be used to determine the presence of a nucleic acid. Labels include, but are not limited to, fluorophores, radioisotopes, chromogens, enzymes, antigens, heavy metals, dyes, magnetic moieties, phosphorescence groups, chemiluminescent groups, and electrochemical detection moieties.

The terms “tag” and “tag complement,” as used herein, mean single-stranded nucleic acids that complement another single-stranded nucleic acid. In certain embodiments, the term “tag complement” means a nucleic acid that is complementary to the nucleic acid designated as the “tag.”

The term “extension assay” means an assay in which nucleotides are added to a nucleic acid one at a time, resulting in a longer nucleic acid. An exemplary extension assay is one that employs a polymerase to add nucleotides. An example of an extension assay is PCR.

The term “hybridization assay” means an assay in which the presence or absence, or amount of one or more hybridized nucleic acid sequences is determined. Such determination may be accomplished either directly or indirectly. A non-limiting example of a hybridization assay is a Southern or Northern blot. Another non-limiting example of a hybridization assay is a ligation assay in which two probes are ligated together if they both hybridize to a target nucleic acid.

The term hybridization assay does not include extension assays. However, a hybridization assay may be employed in a procedure or assay format that also includes an extension assay. Thus, for example, the term hybridization assay does not include PCR, but does include assays in which PCR is subsequently used to determine whether hybridization occurred. A non-limiting example of such a hybridization assay that includes a subsequent extension reaction is OLA-PCR. In certain such embodiments, probes of a ligation probe set are ligated together to form a ligation product if they hybridize to a target nucleic acid. Then, that ligation product may be involved in a subsequent PCR. See, e.g., PCT International Application Nos. WO 01/092579 A3, published Dec. 6, 2001, WO 03/054511 A2, published Jul. 3, 2003, and WO 2004/040020 A1, published May 13, 2004; and U.S. Patent Application Publication Nos. US 2003/0190646 A1, published Oct. 9, 2003, and US 2004/0121371 A1, published Jun. 24, 2004.

In certain embodiments, a hybridization assay may occur subsequent to an extension assay. For example, a hybridization assay in which a probe and/or a target nucleic acid comprises a PCR product is included within the definition of a hybridization assay.

The term “hybridization based separation assay” means a hybridization assay in which a nucleic acid may be at least partially separated from a sample based on its ability to hybridize to one or more other nucleic acids. A non-limiting example of a hybridization based separation assay is an assay in which a sample containing nucleic acids is contacted with a probe that is attached to a solid support.

The term “immobilization hybridization assay” means a hybridization assay in which a nucleic acid has been immobilized on a solid support. In certain embodiments, the immobilized nucleic acid may be a probe or a target nucleic acid. In certain embodiments, the nucleic acid may be immobilized directly to a solid support. In certain embodiments, the nucleic acid may a immobilized indirectly to a solid support. A non-limiting example of an immobilization hybridization assay is a Southern blot.

An “array” is a solid support on which two or more nucleic acids are immobilized. In certain embodiments, arrays are used for any identification or separation of nucleic acids. Examples include, but are not limited to, nucleic acid expression analysis, identification of tagged molecules in a sample, and separation of tagged molecules out of a sample. In certain embodiments, an array is a nucleic acid microarray.

The term “array assay” means a hybridization assay that employs an array. Array assays include, but are not limited to, hybridization based separation assays that employ an array.

The term “feature” means a single location on an array. In certain embodiments, a single feature may have probes that all have the same sequence. In certain embodiments, a single feature may include probes with different sequences.

An “array probe” is a nucleic acid that is immobilized on the solid support of an array, and that is capable of binding to a complementary sequence. In certain embodiments, the sequence complementary to the array probe is a target. The array probe may include Watson-Crick bases and/or modified bases. Modified bases include, but are not limited to, the AEGIS bases (from Eragen Biosciences), which have been described, e.g., in U.S. Pat. Nos. 5,432,272; 5,965,364; and 6,001,983. Additionally, bases may be joined by a natural phosphodiester bond or a different chemical linkage. Different chemical linkages include, but are not limited to, a peptide bond or a Locked Nucleic Acid (LNA) linkage, which is described, e.g., in published PCT applications WO 00/56748; and WO 00/66604. The sequence of the array probe and that of the target may or may not be known. The length of the array probe may be longer, shorter, or the same length as the target.

The term “capture moiety” means any molecule that can be used to at least partially isolate a nucleic acid. In certain embodiments, the term “capture moiety” includes affinity sets.

As used herein, an “affinity set” is a set of molecules that specifically bind to one another. Affinity sets include, but are not limited to, biotin and avidin, biotin and streptavidin, receptor and ligand, antibody and ligand, antibody and antigen, and a polynucleotide sequence and its complement. One or more members of an affinity set may be coupled to a solid support. Exemplary solid supports include, but are not limited to, agarose, sepharose, magnetic beads, polystyrene, polyacrylamide, glass, membranes, silica, semiconductor materials, silicon, and organic polymers.

The term “hybridization indicator assay” means a hybridization assay in which the presence or absence of hybridization is detected by the presence or absence of a signal from a hybridization indicator.

A “hybridization indicator” is any molecule or group of molecules that can be used to determine the presence hybridization. Hybridization indicators produce a detectably different signal if hybridization has occurred than if hybridization has not occurred. The skilled artisan will appreciate that many hybridization indicators may be used in certain embodiments. In certain embodiments, hybridization indicators comprise at least one label and/or tag.

According to certain embodiments, a hybridization indicator may be a “molecular beacon,” which comprises a fluorescent molecule attached to a fluorescence-quenching molecule by an oligonucleotide. When bound to a polynucleotide as double-stranded nucleic acid, the quenching molecule is spaced apart from the fluorescent molecule, and the hybridization indicator may give a fluorescent signal. When the molecular beacon is single-stranded, the oligonucleotide portion can bend flexibly, and the fluorescence-quenching molecule can quench the fluorescent molecule, reducing the amount of fluorescent signal. Certain such systems are described, e.g., in U.S. Pat. No. 5,723,591.

According to certain embodiments, a hybridization indicator is a “nucleic acid binding molecule” that binds or interacts, with nucleic acid. Exemplary interactions include, but are not limited to, ionic bonds, hydrophobic interactions, covalent interactions, complex formation with the minor groove of double-stranded nucleic acid, nucleic acid hybridization, and intercalation. In certain embodiments, such hybridization indicators are molecules that interact with double-stranded nucleic acid.

In certain embodiments, hybridization indicators are “intercalating dyes,” which are molecules that exhibit a detectably different signal when they are intercalated with double-stranded nucleic acid than when they are not intercalated with double-stranded nucleic acid.

In certain embodiments, hybridization indicators are “minor groove binding dyes” that may bind to the minor groove of double-stranded nucleic acid.

In certain embodiments, a nucleic acid binding molecule is a fluorescent dye or other fluorescent molecule that can be excited to fluoresce by specific wavelengths of light, and then fluoresce in another wavelength. According to certain such embodiments, nucleic acid binding molecules may include, but are not limited to, acridine orange; ethidium bromide; thiazole orange; pico green; chromomycin A3; SYBR® Green I (see U.S. Pat. No. 5,436,134); quinolinium, 4-[(3-methyl-2(3H)-benzoxazolylidene)methyl]-1-[3-(trimethylammonio) propyl]-, diiodide (YOPRO®); and quinolinium, 4-[(3-methyl-2(3H)-benzothiazolylidene)methyl]-1-[3-(trimethylammonio)propyl]-, diiodide (TOPRO®). SYBR® Green I, YOPRO®, and TOPRO® are available from Molecular Probes, Inc., Eugene, Oreg.

“Detectably different signal” means that detectable signals from different labels are distinguishable from one another by at least one detection method.

The term “detectable signal value” means a value of the signal that is detected from a label. In certain embodiments, the detectable signal value is the amount or intensity of signal that is detected from a label. Thus, if there is no detectable signal from a label, its detectable signal value is zero (0). In certain embodiments, the detectable signal value is a characteristic of the signal other than the amount or intensity of the signal, such as the spectra, wavelength, color, or lifetime of the signal.

“Detectably different signal value” means that one or more detectable signal values are distinguishable from one another by at least one detection method.

The term “threshold difference between detectable signal values” means a set difference between a first detectable signal value and a second detectable signal value that results when the target nucleic acid that is being sought is present in a sample, but that does not result when the target nucleic acid is absent. In certain embodiments, in which a hybridization indicator is employed the first detectable signal value of a hybridization indicator is the detectable signal value from the hybridization indicator when it is not exposed to double-stranded nucleic acid. The second detectable signal value is detected during and/or after the addition, creation, or amplification of a complementary nucleic acid.

In certain embodiments, the term “to a measurably lesser extent” encompasses situations in which the event in question is reduced at least 10 fold. In certain embodiments, the term “to a measurably lesser extent” encompasses situations in which the event in question is reduced at least 100 fold.

In certain embodiments, a statement that a component may be, is, or has been “substantially removed” means that at least 90% of the component may be, is, or has been removed. In certain embodiments, a statement that a component may be, is, or has been “substantially removed” means that at least 95% of the component may be, is, or has been removed.

The term “amplification product” as used herein means the product of an amplification reaction including, but not limited to, primer extension, the polymerase chain reaction, RNA transcription, and the like. Thus, exemplary amplification products may comprise at least one of primer extension products, PCR amplicons, RNA transcription products, and the like.

A “ligation probe set”, as used herein, is a group of two or more probes designed to detect at least one target. As a non-limiting example, a ligation probe set may comprise two nucleic acid probes designed to hybridize to a target such that, when the two probes are hybridized to the target adjacent to one another, they are suitable for ligation together.

When used in the context of the present invention, “suitable for ligation” means at least one first target-specific probe and at least one second target-specific probe, each comprising an appropriately reactive group. Exemplary reactive groups include, but are not limited to, a free hydroxyl group on the 3′ end of the first probe and a free phosphate group on the 5′ end of the second probe. Exemplary pairs of reactive groups include, but are not limited to: phosphorothioate and tosylate or iodide; esters and hydrazide; RC(O)S⁻, haloalkyl, or RCH₂S and α-haloacyl; thiophosphoryl and bromoacetoamido groups. Exemplary reactive groups include, but are not limited to, S-pivaloyloxymethyl-4-thiothymidine. Additionally, in certain embodiments, first and second target-specific probes are hybridized to the target sequence such that the 3′ end of the first target-specific probe and the 5′ end of the second target-specific probe are immediately adjacent to allow ligation.

A “ligation agent”, as used herein, may comprise any number of enzymatic or chemical (i.e., non-enzymatic) agents that can effect ligation of nucleic acids to one another.

B. Certain Exemplary Embodiments

In certain embodiments, one or more stringency modifier are used in a hybridization assay. In certain embodiments, the stringency modifier comprises 2-Pyrrolidinone. In certain embodiments, the stringency modifier comprises an N-alkyl-pyrrolidinone. In certain embodiments, the stringency modifier comprises delta-valerolactam. In certain embodiments, the stringency modifier comprises epsilon-caprolactam. In certain embodiments, the stringency modifier comprises one or more compounds that is structurally related to 2-Pyrrolidinone, N-alkyl-pyrrolidinone, delta-valerolactam, or epsilon-caprolactam. In certain embodiments, one or more stringency modifiers of the present invention are used in a hybridization that also comprises one or more other stringency modifiers. In certain embodiments, one or more stringency modifiers of the present invention are used in a hybridization that comprises formamide.

1. Certain Aspects

In certain embodiments, the stringency modifiers of the present invention are used to increase stringency in a hybridization. In certain embodiments, the stringency modifiers of the present invention are used in a hybridization solution in place of formamide. In certain embodiments, the stringency modifiers of the present invention have certain advantages over formamide, which has been used to increase stringency. In certain embodiments, the stringency modifiers or the present invention are more chemically stable in aqueous solutions than formamide. When exposed to water, formamide typically undergoes hydrolytic break down into ammonium and carbonate ions. That breakdown typically is accelerated by high temperatures, acids, and bases. In certain embodiments, the stringency modifiers of the present invention are more stable in water. Thus, in certain embodiments, aqueous, premixed hybridization buffers containing stringency modifiers of the present invention have a longer shelf life than those made with formamide. As a result, in certain embodiments, one may not have to add the stringency modifier to the buffer on the day of use, which often is the case with formamide. In addition, in certain embodiments, premixed solutions result in more consistent batch to batch uniformity than hybridization buffers that are mixed on each day of use, thereby reducing variability of hybridization assay conditions. In certain embodiments, variability is also reduced because certain stringency modifiers of the present invention are less likely than formamide to break down during the hybridization assay, resulting in more consistent concentration of the stringency modifier throughout the duration of the assay.

Certain stringency modifiers of the present invention are less hazardous than formamide, which is both a poison and a teratogen. Further, because formamide is usually used neat (in its pure form), the exposure to users may be substantial. Certain stringency modifiers of the present invention are less toxic and/or less teratogenic than formamide. Furthermore, usually, premixed hybridization buffers reduce or eliminate the user's exposure to the neat form of the stringency modifier. Thus, certain embodiments reduce the hazard to those performing hybridization assays.

2. Certain Exemplary Assays

In certain embodiments, at least one of the chemical stringency modifiers of the present invention is added to a hybridization buffer at a concentration from about 1% to about 30% (calculated as the volume of the stringency modifier/total volume(v/v)). In certain embodiments, at least one of the chemical stringency modifiers of the present invention is added to a hybridization buffer at a concentration from about 1% to about 10%. In certain embodiments, at least one of the chemical stringency modifiers of the present invention is added to a hybridization buffer at a concentration from about 5% to about 10%.

In certain embodiments, the hybridization buffer comprises other components, e.g., one or more of the following: salts (including, but not limited to, sodium chloride), buffer ions (including, but not limited to, phosphate and morpholinoethanesulfonate), detergents (including, but not limited to, sodium dodecylsulphate, triton X100, Tween-20, and sodium lauroylsarcosine), and blocking agents (including, but not limited to, polyAdenosine, tRNA, and herring sperm DNA).

In certain embodiments, the concentration of one or more stringency modifier of the present invention may be varied to achieve a desired level of stringency. One of skill in the art will recognize that in certain embodiments, the concentration of components of a hybridization solution, including a stringency modifier, may be manipulated to optimize a desired result.

Exemplary assays for which the stringency modifiers of the present invention may be used include, but are not limited to: Southern blots (see, e.g., Sambrook and Russell, Molecular Cloning A Laboratory Manual, pp 6.33-6.58 (3^{rd} Edition, 2001)); Northern blots (see, e.g., id., at 7.18-7.42); dot blots (see, e.g., id., at 7.46-7.51); slot blots (see e.g., id.); ligation assays, including without limitation, oligonucleotide ligation assays (OLA), OLA-PCR assays (see, e.g., U.S. Pat. No. 5,912,148); arrays, including without limitation, microarrays; any assay that uses a tag sequence.

In certain embodiments, at least one stringency modifier of the present invention is used in a hybridization assay. In certain embodiments, one or more stringency modifier of the present invention is used in a hybridization based separation assay. In certain embodiments, one or more stringency modifier of the present invention is used in an immobilization hybridization assay. In certain embodiments, one or more stringency modifier of the present invention is used in an array assay. In certain embodiments, one or more stringency modifier of the present invention is used in a hybridization indicator assay.

In certain embodiments, at least one stringency modifier of the present invention is used in an array assay. In certain embodiments, a sample that may comprise a target nucleic acid and a hybridization buffer comprising at least one of the stringency modifiers of the present invention are exposed to the surface of an array. In certain embodiments, the array is planar. In certain embodiments, the array is non-planar, e.g., an optical fiber bundle, a bead, or other solid surface.

In certain embodiments, the surface of an array comprises probes immobilized to different features. In certain embodiments, the sample that may include target and the hybridization buffer containing at least one of the stringency modifiers of the present invention are incubated with the probes of the array for about 3 hours to about 24 hours at a temperature of about 35° C. to about 80° C. In certain such embodiments, after incubation, the array surface may be washed with a dilute buffer and/or water to remove unbound sample. In certain embodiments, the array is dried. According to various embodiments, the array is analyzed by any of a variety of methods.

In certain embodiments, the target nucleic acid used in an array assay comprises a label. Exemplary labels include, but are not limited to, fluorophores. In certain embodiments, the presence on an array of target nucleic acid comprising fluorophores is detected using a scanner. In certain embodiments, the scanner employs a laser for excitation and a filtered emission photo-multiplier tube (PMT) device for detection. Exemplary scanners are commercially available, e.g., from Axon Instruments Inc. (Union City, Calif.), from Agilent Technologies (Palo Alto, Calif.), and from Genomic Solutions (Ann Arbor, Mich.). In certain such embodiments, the excitation laser beam moves over each feature of the surface of the array. In certain embodiments, target nucleic acid comprising a fluorophore emits a signal upon excitation by the excitation laser beam. In certain embodiments, the intensity of the emitted signal is detected by a PMT device. In certain embodiments, signal detected at a feature indicates the presence of target nucleic acid at that feature. Presence of target nucleic acid at a particular feature indicates hybridization of target nucleic acid to the corresponding probe.

In certain embodiments, the emitted signal is analyzed by software (including, but not limited to Agilent Technologies, GenePix Pro, and Axon Instruments Inc.). In certain embodiments, such software is used to determine the intensity of the signal for a feature to determine whether or not target nucleic acid hybridized to particular probes.

In certain embodiments, in situ spotted arrays comprising features to which various array probes had been immobilized can be used. In certain embodiments, array probes can have a length of anywhere from about 25 nucleotides to about 60 nucleotides. In certain embodiments, the array probes can be 25 nucleotides long. In certain embodiments, the array probes can be 60 nucleotides long.

In certain embodiments, DNA target nucleic acids comprising Cy3 or Cy5 labels can be used in hybridization assays with in situ spotted arrays. In certain embodiments, hybridization solution can comprise the labeled target nucleic acid, 50 mM MES (pH 6.5) (Sigma Chemicals, Inc., cat# M-528), 0.5% Sarkosyl (Sigma Chemicals, Inc., cat# L9150), 1.0 M NaCl (EM Science, cat# 7710), 1 mg/ml tRNA (Sigma Chemicals, Inc., cat# R4018), and 0.5 mg/ml poly A (Amersham Pharmacia Inc., cat# 27-4110-01), and either 5%,10%, or 20% 2-pyrrolidinone. In certain embodiments, the arrays can be incubated in one of the hybridization solutions (each with a different concentration of 2-pyrrolidinone) for 18 hours at 55° C.

In certain embodiments, following incubation, the arrays can be removed from the hybridization solutions and can be washed with a solution of 6×SSC and 0.005% Triton-102 for 10 minutes at room temperature with constant stirring, and then in a solution of 0.1×SSC for 5 minutes at room temperature with constant stirring. In certain embodiments, after washing, the arrays can be spun in a centrifuge at 400 rpm for 5 minutes to dry them.

In certain embodiments, the arrays can then be scanned using an Agilent Scanner (Agilent Technologies, Palo Alto, Calif., Catalog No. G2565BA) to detect the presence or absence of label at each feature.

In certain embodiments, at least one stringency modifier of the present invention is used in a hybridization assay in which a hybridization indicator is included in the hybridization assay. According to certain embodiments, such hybridization indicator indicates the amount of double-stranded nucleic acid and thus, can be used to assess the amount of hybridized product present. In certain embodiments, the hybridization indicator is a nucleic acid binding molecule that binds with the double-stranded nucleic acid, resulting in a change in its fluorescent qualities from when it is not bound to double-stranded nucleic acid. Exemplary hybridization indicators of this type include, but are not limited to, dyes including, but not limited to, acridine orange, ethidium bromide, and SYBR® Green I (Molecular Probes, Inc.) (see, e.g., U.S. Pat. No. 5,436,134).

In certain embodiments, a hybridization indicator comprises a label that is connected to a quenching molecule by a specific oligonucleotide. Such hybridization indicators include, but are not limited to, molecular beacons. Examples of certain hybridization indicators and molecular beacons are described, e.g., in U.S. Pat. Nos. 5,538,848 and 5,723,591.

In certain embodiments, the amount of hybridization indicator that gives a fluorescent signal typically relates to the amount of double-stranded nucleic acid present. In certain such embodiments, one can therefore determine the amount of hybridized product by measuring the signal value from the hybridization indicator.

According to certain embodiments, one can employ an internal standard to quantify the hybridization product indicated by the fluorescent signal. According to certain embodiments, different hybridization indicators distinguish between different target nucleic acids. A non-limiting example of such a hybridization indicator is a molecular beacon, wherein a fluorescent molecule is attached to a fluorescence-quenching molecule by an oligonucleotide. In certain embodiments, the oligonucleotide portion of the hybridization indicator may bind to a specific target nucleic acid. Different hybridization indicators, comprising different labels fluorescing at different wavelengths, can distinguish between different hybridization products within the same hybridization assay.

4. Certain Exemplary Kits

In certain embodiments, kits are provided that are designed to expedite performing certain methods. In certain embodiments, kits serve to expedite the performance of the methods of interest by assembling two or more components used in carrying out the methods. In certain embodiments, kits may contain components in pre-measured unit amounts to minimize the need for measurements by end-users. In certain embodiments, kits may include instructions for performing one or more methods. In certain embodiments, the kit components are optimized to operate in conjunction with one another.

In certain embodiments, kits comprise reagents for use in a hybridization assay. In certain embodiments, kits comprise at least one stringency modifier of the present invention. In certain embodiments, such kits comprise a hybridization buffer which comprises at least one stringency modifier of the present invention.

In certain embodiments, kits comprise reagents useful for performing an array assay. In certain embodiments, such kits comprise at least one stringency modifier of the present invention. In certain embodiments, such kits comprise a hybridization buffer which comprises at least one stringency modifier of the present invention. In certain embodiments, such kits comprise a planar array. In certain embodiments, such kits comprise a non-planar array.

1. A method of modifying stringency in a hybridization assay comprising, incubating nucleic acids in the presence of at least one stringency modifier, wherein the at least one stringency modifier is selected from: 2-pyrrolidinone; an N-alkyl-pyrrolidinone; delta-valerolactam; and epsilon-caprolactam.

2. The method of claim 1, wherein the concentration of the stringency modifier is about 1% to about 30% (v/v)

3. The method of claim 2, wherein the stringency modifier is 2-pyrrolidinone.

4. The method of claim 1, wherein the hybridization assay is an array assay.

5. The method of claim 4, wherein the concentration of the stringency modifier is about 1% to about 30% (v/v).

6. The method of claim 5, wherein the stringency modifier is 2-pyrrolidinone.

7. The method of claim 1, wherein the hybridization assay is a hybridization based separation assay.

8. The method of claim 7, wherein the concentration of the stringency modifier is about 1% to about 30% (v/v).

9. The method of claim 8, wherein the stringency modifier is 2-pyrrolidinone.

10. The method of claim 1, wherein the hybridization assay is an immobilization assay.

11. The method of claim 10, wherein the concentration of the stringency modifier is about 1% to about 30% (v/v).

12. The method of claim 11, wherein the stringency modifier is 2-pyrrolidinone.

13. The method of claim 1 wherein the hybridization assay is a hybridization indicator assay.

14. The method of claim 13, wherein the concentration of the stringency modifier is about 1% to about 30% (v/v).

15. The method of claim 13, wherein the stringency modifier is 2-pyrrolidinone.

16. The method of claim 1, wherein the hybridization assay comprises a labeled probe and/or a labeled target.

17. A kit for performing an array assay comprising: (i) a hybridization buffer comprising at least one stringency modifier selected from: 2-pyrrolidinone; an N-alkyl-pyrrolidinone; delta-valerolactam; and epsilon-caprolactam; and (ii) an array.
